(19) United States
(12) Patent Application Publication
(10) Pub. No.: US 2026/0146231 A1
(43) Pub. Date: May 28, 2026

(54) BRAIN ORGANOIDS, METHOD OF PRODUCTION, AND METHOD OF USE

(71) Applicant: THE BOARD OF TRUSTEES OF THE LELAND STANFORD JUNIOR UNIVERSITY, Stanford, CA (US)

(72) Inventor: Sabra Djomehri, Los Altos, CA (US)

(21) Appl. No.: 19/400,035

(22) Filed: Nov. 25, 2025

Related U.S. Application Data
(60) Provisional application No. 63/725,256, filed on Nov. 26, 2024.

(51) Int. Cl. C12N 5/0797; G01N 33/50

(52) U.S. Cl. CPC C12N 5/0623; G01N 33/5082; C12N 2500/38; C12N 2501/11; C12N 2501/115; C12N 2501/13; C12N 2501/33; C12N 2501/727; C12N 2503/04; C12N 2533/78; C12N 2533/90

(57) ABSTRACT

The disclosure provides a method for producing brain organoids, brain organoids produced by the method, and use of the brain organoids for characterizing the activity of a substance in brain tissue.

### CROSS-REFERENCE TO RELATED APPLICATIONS

This application claims the benefit of priority of U.S. Patent Application. No. 63/725,256, filed Nov. 26, 2024, which is hereby incorporated by reference in its entirety.

### FIELD

The disclosure relates to brain organoids, methods of producing brain organoids, and methods of using brain organoids.

### BACKGROUND

Neurodegenerative diseases such as Alzheimer's disease are characterized by progressive cognitive deterioration and are common in the aging population with a high fatality rate. Current therapies targeting disease lesions, plaques and tangles are greatly lacking, and the complex pathology makes it challenging to model in vitro. This is due to inaccessibility of brain tissue and difficulty recapitulating adult neurogenesis and key targetable phenotypes of Alzheimer's disease. Three-dimensional (3D) organoid models have demonstrated significant potential to assess disease progression and can be utilized as efficient drug screening platforms. However, organoid techniques are currently limited by several constraints, including the lack of efficient nutrient exchange and generation of uniform 3D structures, lack of high throughput screening approaches, and limited reproducibility and scalability for translational applications.

Brain organoids are a particularly new and innovative tool to model neurological diseases given the difficult accessibility of human brain tissue. Current models of neurodegenerative diseases like Alzheimer's disease have relied on animal models and in vitro models derived from human pluripotent stem cells (hPSCs). The animal models are limited by high mortality, poor representation of complex Alzheimer's disease pathophysiology, making it difficult to evaluate the efficacy and mechanisms of novel drugs. Alzheimer's disease iPSC models have shown versatility in many ways, but lack crucial features of plaque formation, early development of tangles, and underlying inflammation. Also, iPSCs do not exist naturally since they are generated from somatic cells by ectopic co-expression of pluripotency factors and have mostly relied on either 2D methods or embryoid body (EB) formation to generate organoids. One major limitation of EB neural induction is that it can lead to low reproducibility and variable outcomes, such as random differentiation into non-neuroectodermal cell types. Furthermore, the translational utility of iPSC Alzheimer's disease models is hindered by long culture times and a high production cost for in vitro generation, which limits their therapeutic potential in performing large-scale drug discovery studies.

### SUMMARY

The disclosure provides a method of generating a brain organoid, the method comprising culturing a neurosphere comprising neural stem cells (NSCs) in a composition comprising from about 0.01 to about 0.1% hydrogel matrix (w/v) and about 1 to about 2% extracellular matrix (ECM) (v/v) in a hanging droplet cell suspension format for a period of time to generate a brain organoid. In various aspects, the hydrogel matrix is methylcellulose and the ECM is Matrigel®, and the composition comprises from about 0.01 to about 0.1% methylcellulose (w/v) and about 1 to about 2% Matrigel (v/v). Optionally, the composition further comprises a growth factor and growth supplement (e.g., insulin). Also optionally, the period of time is at least about 10 days.

In various aspects, the method further comprises, prior to culturing the neurosphere in the composition, an induction step comprising culturing NSCs in the presence of a ROCK inhibitor (ROCKi) and an N-2 supplement to produce a neurosphere. Optionally, the induction step comprises culturing the NSCs in the presence of Neurobasal A-Medium, about 2% B- 27 supplement (v/v), about 1% PSA (v/v), Glutamax, about 0.5 to about 1% N-2 supplement (v/v), and/or about 0.1 to about 0.5% ROCK inhibitor (v/v). The induction step may be performed for about 10 to about 12 days.

In various aspects, the method further comprises a maturation step comprising culturing the brain organoid in the presence of vitamin A, a neurotrophic factor, and a growth factor. Optionally, the maturation step comprises culturing the brain organoid in the presence of Neurobasal-A Medium, about 2% B-27 supplement with vitamin A (v/v), about 1% PSA (v/v), Glutamax, about 0.5% N-2 supplement (v/v), about 0.2% EGF and bFGF (w/v), about 0.05 to about 0.1% NT- 3 and/or BDNF (w/v), and/or about 0.05% growth supplement (e.g., insulin) (v/v). Also optionally, the maturation step may be performed for at least about 10 days.

The disclosure further provides a method of generating a brain organoid, the method comprising: (i) an induction step comprising culturing adult neural stem cells (NSCs) derived from a subventricular zone (SVZ) in an induction medium comprising a ROCK inhibitor (ROCKi), an N-2 supplement, and a growth factor for a first period of time to produce a neurosphere; (ii) an expansion step comprising culturing the neurosphere in an expansion medium comprising hydrogel matrix, extracellular matrix (ECM), an N-2 supplement, a growth factor, and growth supplement (e.g., insulin) in a hanging droplet suspension format for a second period of time to produce a brain organoid; and (iii) a maturation step comprising culturing the brain organoid in a maturation medium comprising vitamin A, N-2 supplement, a growth factor, a neurotrophic factor, and growth supplement (e.g., insulin) for a third period of time.

Further provided is a brain organoid produced by the method described herein. The brain organoid optionally displays a diameter of about 1 mm and/or optionally expresses SOX2, Nestin, GFAP, DCX, MAP2, NeuN, CTIP2, and/or Ki67. A method of characterizing the activity of a substance in brain tissue using the brain organoid produced as described herein, wherein the substance is contacted with the organoid and the biological effect of the substance on the brain organoid is characterized, also is provided.

Additional embodiments and aspects of the presently disclosed compositions and methods are provided below. All headings are simply for organization and are not intended to limit the disclosure in any manner. The content of any individual section may be equally applicable to all sections.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more. As such, the terms “a” (or “an”), “one or more,” and “at least one” can be used interchangeably herein. The term “or” should be understood to encompass items in the alternative or together, unless context unambiguously requires otherwise.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. However, the description also contemplates the same ranges in which the lower and/or the higher endpoint is excluded. When the term “about” is used, it means the recited number plus or minus 5%, 10%, or more of that recited number. The actual variation intended is determinable from the context.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. Only such limitations which are described herein as critical to the invention should be viewed as such; variations of the invention lacking limitations which have not been described herein as critical are intended as aspects of the invention.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document.

### BRIEF DESCRIPTION OF THE DRAWINGS

FIG. 1 depicts a representative protocol of the disclosure for generating a brain organoid from neural stem cells (NSCs) harvested from the subventricular zone (SVZ) of a subject. A neurosphere induction phase occurs between about day 0 and day 10-12 and comprises culturing NSCs in, e.g., neurobasal-A media containing epidermal growth factor (EGF), basic fibroblast growth factor (bFGF), N-2 supplement, and a ROCKi (Y-27632 dihydrochloride). In various aspects, the cells are cultured under conditions for neurosphere induction until spheres of about 200 to about 500 μm are achieved. An expansion phase occurs between days 10-12 through days 18-20, wherein the cells are transferred to a hanging droplet array plate (e.g., a 384-well plate). An expansion medium was designed with a unique extracellular matrix composition comprising low concentrations of extracellular matrix (here, Matrigel®) and hydrogel (here, methylcellulose) to mimic the biophysical properties of brain tissue microenvironment. After about 72 hours, the Matrigel® is removed and media refreshed periodically. Around day 18-20, a maturation phase is employed to produce matured organoids. Optionally, the transition from expansion phase to maturation phase is triggered by appearance of neuropithelium (e.g., appearance of neuroepithelial budding) and/or increased diameter of the cellular structure (e.g., about 600 to about 900 μm). During the maturation phase, the amount of N-2 supplement and growth factor in the media is reduced, and optionally vitamin A and neurotrophic factors (e.g., neurotrophin-3 (NT-3) and brain-derived neurotrophic factor (BDNF)) are introduced.

FIG. 2 depicts representative media components for use in the method disclosed herein. The disclosure is not limited to the particular media represented in the figure; the schematic is provided merely to illustrate an embodiment of the disclosure.

FIG. 3 is an illustration of a representative humidification chamber which may be used during the expansion and/or maturation phase of the method described herein.

FIG. 4 is an image of a brain organoid produced using the method of Example 1, demonstrating that the organoid recapitulates a rudimentary neuroepithelial architecture. The image is presented alongside a schematic of a single rosette structure with organization of progenitors and neurons. The image was produced by immunofluorescence imaging of brain organoids (10× magnification, scale bar=100 μm) with positive expression for both stem cells (Sox2) and mature neuronal markers (Map2), demonstrating a typical pattern of cortical rosettes found in the neuroepithelium.

FIG. 5 is a graph illustrating the average diameter (μm; y-axis) of NSC-derived 3D structures over time (days; x-axis), wherein the NSCs are embryonic mouse NSCs (circles), adult mouse NSCs (squares), and human pluripotent stem cells (hPSCs, triangles). The adult NSCs derived from SV and cultured using the method disclosed (FIG. 5, squares) required reduced culture time to achieve a desired size compared to iPSC-derived brain organoids cultured in previous methodologies (FIG. 5, triangles). Previous methodologies included culture in growth media comprising Neurobasal-A (Invitrogen) supplemented with Glutamax (Life Technologies), 2% B 27-A (Invitrogen), mouse recombinant epidermal growth factor (EGF; 20 ng/ml) and basic fibroblast growth factor (bFGF; 20 ng/ml) (Shenandoah Biotechnology).

FIGS. 6A-6C pertain to testing of various concentrations of methylcellulose and Matrigel® on organoid formation. FIG. 6A is an image of organoids produced under different conditions which resulted in irregular structures, failed structures, or successful organoid production. FIGS. 6B and 6C are graphs illustrating the effects of varying amounts of hydrogel (here, methylcellulose) (FIG. 6B) or extracellular matrix (here, Matrigel®) (FIG. 6C) on organoid formation (y-axis). With respect to FIGS. 6B and 6C, the categorization of “successful,” “irregular,” or “failed” 3D cultures was determined based on morphological features using brightfield images for ˜10-20 structures per category. Successfully formed 3D structures were defined as >500 μm with well-defined periphery, evidence of expansion, opacity, and budding. Failed structures were designated using the following criteria: failure of aggregation or sphere formation, small structures without evidence of growth and lack of differentiation, irregular shape with unclear borders, abnormal periphery, and accumulation of cellular debris or cell death. Irregular structures were those which exhibit both successful and failed features, but were ultimately not selected for downstream analysis.

FIGS. 7A and 7B are graphs illustrating neurosphere induction efficiency (FIG. 7A) and organoid-forming efficiency (FIG. 7B) produced using the methods set forth in Example 3. Neurosphere induction efficiency measures the capability of spheres to undergo expansion in 3D culture, and organoid-forming efficiency is an overall metric of successful organoid formation through the maturation phase. The “preliminary” method referenced employed the neurosphere media described in Reinitz et al, Elife. 2022 Mar. 21:11:e66037 (i.e., employing growth media comprising Neurobasal-A (Invitrogen) supplemented with Glutamax (Life Technologies), 2% B27-A (Invitrogen), mouse recombinant epidermal growth factor (EGF; 20 ng/ml) and basic fibroblast growth factor (bFGF; 20 ng/ml) (Shenandoah Biotechnology). The “improved” protocol describes the combinatorial use of supplements ROCKi, N2, insulin, vitamin A, the amounts of methylcellulose and Matrigel® described herein, as well as EGF, bFGF and neurotrophic factors BDNF and NT-3.

### DETAILED DESCRIPTION

Organoids are an in vitro generated mass of cells or tissue that mimics organ structure and function. Brain organoids are three-dimensional (3D) structures derived from stem cells that reflect early brain organization. The present disclosure is based, at least in part, on the development of a method for producing brain organoids which more accurately reflects the structure and physiology of brain tissue than previous methods. The materials and methods described herein represent an advancement in the art by, e.g., offering a platform for consistently producing high quality brain organoids suitable for high throughput screening of therapeutic candidates for neural diseases, including Alzheimer's disease.

In one aspect, the disclosure provides a method of generating a brain organoid. The method comprises culturing a neurosphere comprising neural stem cells (NSCs) in a composition comprising a hydrogel matrix and an extracellular matrix in a hanging droplet cell suspension format for a period of time to generate a brain organoid. Merely for ease of reference herein, this aspect of the method is also referred to herein in terms of “expansion phase.” The combination of cell type, matrix combination, and hanging droplet cell suspension format results in particularly high quality brain organoids.

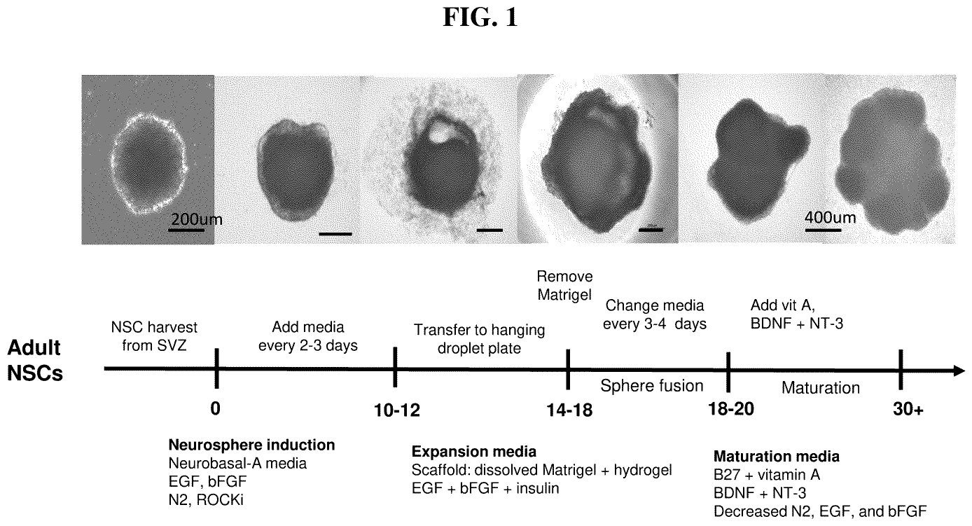

Neural stem cells (NSCs) are well characterized in the art. In various aspects of the disclosure, the NSCs used in the instant methods are adult NSCs, optionally derived from a subventricular zone (SVZ) of the brain. Adult NSCs may be identified by, e.g., stem cell markers SOX2, GFAP, and/or Nestin and absence of immature neuronal marker such as DCX; mature markers Map2 and/or NeuN; and a deep cortical layer marker (such as BCL11B or Ctip2) expressed with correct radial polarity patterns. Optionally, the NSCs are derived from a subject suffering from a neurological disease or disorder, such as Alzheimer's disease. The “subject” may be any mammal, including, but not limited to, a human or non-human mammal, such as a bovine, equine, canine, ovine, rodent, or feline subjects. In various aspects, the NSCs express Sox2, Nestin, and GFAP and are present within a neurosphere. Neurospheres are free-floating clusters of neural stem cells which form a 3D structure, as illustrated in FIG. 1. Neurospheres are generally an aggregate or cluster of either NSCs or neural progenitor cells (NPCs), optionally grown in a 3D culture/suspension system, which do not express (or minimally express) intermediate or mature markers. Neurospheres, in some aspects, without the necessary stimuli, are not capable of self-assembly and expansion into more complex tissue-like structures, such as those that mimic the structure and function of organs. The morphological features of neurospheres include, e.g., small diameter (<500 μm), dark opacity, and no budding.

In instances when the NSCs transferred from an induction medium to the hanging drop suspension format for expansion, the neurospheres achieved in the induction phase are preferably transferred directly to droplets, without further passaging or dissociating the neurospheres. In this regard, in various aspects of the method, neurospheres applied to the hanging droplet suspension format are at least 100 μm in size (e.g., between about 100 μm and 500 μm in size). Optionally, the neurospheres are about 100 μm to about 300 μm, about 100 μm to about 200 μm, about 200 μm to about 300 μm in size, or about 200 μm to about 400 μm in size. In an aspect of the disclosure, the neurospheres applied in the hanging drop suspension format are about 200 μm to about 300 μm in size.

The method comprises culturing NSCs in a composition comprising a hydrogel matrix and an extracellular matrix. Examples of hydrogel matrices include, but are not limited to, methylcellulose and alginate and combinations thereof. Examples of extracellular matrix components include, but are not limited to, Matrigel®, collagen, and laminins. The combination of hydrogel and extracellular matrix components produce a biomimetic scaffold for cells to differentiate into tissue-like 3D structures. In various aspects, the extracellular matrix component is Matrigel® and/or the hydrogel matrix is methylcellulose. In various aspects of the disclosure, the NSCs are cultured in a scaffold comprising Matrigel® and methylcellulose.

The amounts of hydrogel matrix and extracellular matrix present in the expansion medium provide a scaffold component of the medium which mimics the brain microenvironment, resulting in brain organoids that more closely mimic native tissue. In various aspects, the composition comprises from about 0.01% to about 0.1% hydrogel matrix (e.g., methylcellulose) (w/v) and about 1% to about 2% extracellular matrix component (e.g., Matrigel®) (v/v). For instance, the composition may comprise about 0.01% to about 0.05% (v/v), about 0.01% to about 0.03% (v/v), 0.02% to about 0.08% (v/v), or about 0.02% to about 0.05% (v/v) of hydrogel matrix, optionally methylcellulose. In some aspects, the composition comprises about 0.01%, about 0.02%, about 0.03%, about 0.04%, about 0.05%, about 0.06%, about 0.07%, about 0.08%, about 0.09%, or about 0.1% hydrogel matrix (e.g., methylcellulose) (w/v). In some aspects, the composition comprises about 0.5% to about 3% (v/v) extracellular matrix component, e.g., about 0.75% to about 2.5% (v/v), about 0.5% to about 2% (v/v), or about 1% to about 2.5% (v/v). For instance, the composition comprises about 1%, about 1.1%, about 1.2%, about 1.3%, about 1.4%, about 1.5%, about 1.6%, about 1.7%, about 1.8%, about 1.9%, or about 2% extracellular matrix component (e.g., Matrigel®) (v/v), or any range having these values as endpoints. In aspects of the disclosure, the composition comprises about 0.02% methylcellulose (w/v) and about 1% to about 2% Matrigel (v/v). In aspects of the disclosure, the composition comprises about 0.018% Methocel™ (w/v) and about 1% to about 2% Matrigel (v/v).

In various aspects, the composition further comprises a growth factor and growth supplement (e.g., insulin). Suitable growth factors include, but are not limited to, epidermal growth factor (EGF) and basic fibroblast growth factor (bFGF). Additional contemplated growth factors include, but are not limited to, Vascular endothelial growth factor (VEGF), Glial cell line-derived neurotrophic factor (GDNF), fibroblast growth factor 2 (FGF2s), Bone morphogenetic protein (BMPs), suppressor of Mothers Against Decapentaplegic (SMAD), WNTs, Sonic Hedgehog (SHH), tumor growth factor (TGFs), Rho-associated coiled-coil containing protein kinase (ROCK), mitogen-activated protein kinase (MAPK), and Hepatocyte growth factor (HGF). These growth factors are optionally combined with the supplement B27, with or without retinoic acid. The composition optionally comprises about 0.2% to about 0.8% EGF and bFGF (w/v) (e.g., about 0.2% to about 0.5%, about 0.2% to about 0.4%, about 0.3% to about 0.6%, about 0.4% to about 0.8%, or about 0.6% to about 0.8% EGF and bFGF (w/v)). Growth supplements may include, but are not limited to, a chemical entity that is not derived from serum or biological extracts. In various aspects, the growth supplement is insulin. The composition also optionally comprises about 0.05% to about 0.1% insulin (v/v) (e.g., about 0.05% to about 0.075%, about 0.075% to about 0.1%, or about 0.06% to about 0.08% insulin (v/v)).

Any of the media described herein may comprise a basic medium matrix (i.e., a core medium suitable for culturing neural cells to which various components particular for each step of the method is added). Examples of such medium matrices include, but are not limited to, Neurobasal™ medium (pre-natal and fetal neuronal), Neurobasal-A™ medium (post-natal and adult neuronal), B27™ plus medium, Neurocult™ medium (basal medium for mouse or rat NSC and progenitor cells), and cerebral organoid basal medium (hPSC, iPS, or PSC-derived). In various aspects, the medium comprises Neurobasal A™ medium (available from, e.g., Thermo Fisher). The medium also, in various aspects, may comprise B27 (with or without vitamin A), N-2 supplement, glutamax, and/or Polysialic acid (PSA). A representative basic medium matrix of the disclosure for organoid culture comprises Neurobasal A™ medium, B27 (e.g., about 2% (v/v)), PSA and Glutamax (e.g., about 1% (v/v)), and N-2 supplement (e.g., about 0.5-1% (v/v)).

Optionally, the composition for the expansion phase comprises Neurobasal-A™ medium, B27, N-2 supplement, glutamax, PSA, EGF, bFGF, Matrigel®, methylcellulose, and insulin. A representative formulation comprises 10 ml Neurobasal A™ medium+200 μl B27 (50×), 100 μl PSA, 100 μl Glutamax, 50-100 μl N-2 supplement, 40 μl EGF, 40 μl bFGF, 40 μl insulin, 1%-2% Matrigel, and 0.01%-0.1% methylcellulose (hydrogel).

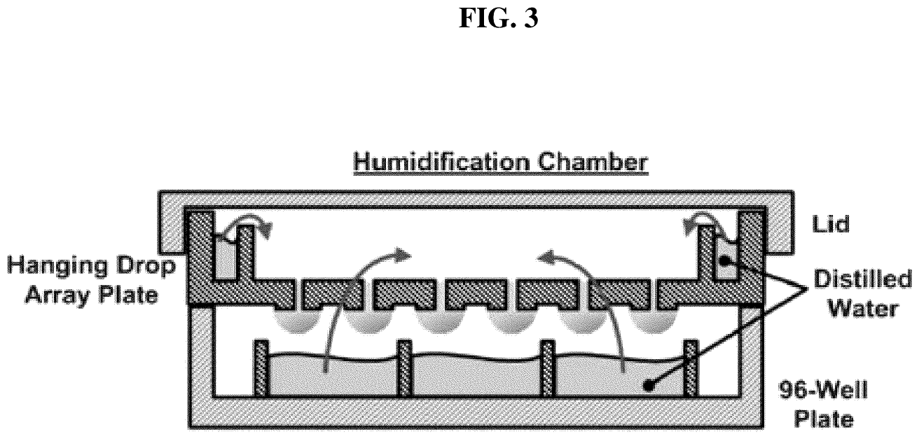

The neurospheres are cultured in a hanging droplet suspension format. Hanging droplet suspension format involves forming droplets of cell suspension which is suspended from a solid support, often a solid surface coated with hydrophilic material, and overlaid on a vessel filled with a liquid capable of humidification of the surrounding environment (e.g., water). Merely to illustrate, a representative hanging droplet suspension system is represented in FIG. 3. In various aspects, the neurospheres are cultured in a hanging droplet suspension in a hanging drop plate coated with hydrophilic material and overlaid on a vessel filled with water. Any system which allows the cell suspension droplets to remain suspended with minimal contact with solid surfaces and allows the neurospheres to expand and develop into organoid structures is suitable for use in the context of the disclosure. In a representative embodiment, the neurospheres are cultured in a 384-well hanging droplet array system coated with hydrophilic coating comprising Pluronic F108 and overlaid on reservoir filled with water may be employed. Optionally, the droplets are applied in a volume of about 20 μl to about 50 ∞l, such as about 25 μl.

A novel aspect of the method described herein is the combination of the hanging droplet platform with the hybrid biomaterial scaffold composite formulation comprising the hydrogel matrix (e.g., methylcellulose) and extracellular matrix (e.g., Matrigel®) present in reduced concentrations (e.g., about 0.02% (w/v) MethoCel™ and 1-2% Matrigen® (v/v)), to closely mimic the biophysical properties of the brain tissue microenvironment. The scaffold composite described herein has not been utilized in neural stem cell culture, and it was surprisingly observed to allow neural stem cells to differentiate and expand in a uniform manner. The reduced concentration of the hydrogel (e.g., methylcellulose at ˜0.02% (w/v) (MethoCel™)) together with relatively low concentration of extracellular matrix (e.g., dissolved Matrigel (1-2% (v/v))) consistently produced remarkably successful organoids (e.g., organoids having a single rosette structure). Without wishing to be bound by any particular theory, the hanging droplet suspension format, in combination with the scaffold composite, allows superior oxygen and transport gradients and improved nutrient exchange. Indeed, the small droplet diffusion kinetics produces an ideal symmetry for both the expansion and maturation phases without the need for additional equipment for agitation of 3D structures, such as using a spinning bioreactor. The resulting organoids also can be easily collected for downstream quantitative testing, such as multi-omics analyses or biochemical assays without requiring complicated procedures, making the system a fast, cost-effective, and highly scalable system for brain organoid production.

The neurospheres are cultured in the expansion medium in a hanging droplet cell suspension format for a period of time to generate a brain organoid. In various aspects, the period of time is about six to about 12 days, such as about six days to about 10 days or about eight days to about 12 days, or about 10 days. In various aspects, the extracellular matrix component of the culture medium is removed around 72 hours after the expansion phase begins (e.g., by media exchange of the starting medium with replacement medium which does not comprise extracellular matrix (Matrigel®)). Optionally, the period of time is such that the spheres are about 600-900 μm in size.

In various aspects of the disclosure, the method further comprises, prior to culturing the NSCs in the composition, an induction step comprising culturing NSCs in the presence of a ROCK inhibitor (ROCKi) and an N-2 supplement. In this regard, adult NSCs (e.g., adult NSCs harvested from SVZ) are cultured in an induction medium for a period of time to produce neurospheres. The induction medium does not comprise scaffold components (i.e., does not comprise hydrogel or extracellular matrix components). The induction media may comprise a medium matrix suitable for culturing neural cells, such as any of the media described herein (e.g., Neurobasal A™M), including a ROCKi and an N-2 supplement. Optionally, the ROCKi is Y-27632 ((1R,4r)-4-((R)-1-Aminoethyl)-N-(pyridin-4-yl)cyclohexanecarboxamide). In various aspects, the medium further comprises a growth factor, such as EGF and/or bFGF (e.g., EGF and bFGF, such as about 0.4% to about 1% (v/v) or about 0.6% to about 0.9% (v/v) or about 0.8% (v/v) EGF and bFGF). Optionally, the induction step comprises culturing the NSCs in the presence of Neurobasal A™ medium, B-27 supplement (e.g., about 2% (v/v)), PSA (e.g., about 1% (v/v)), Glutamax (e.g., about 1% (v/v)), N-2 supplement (e.g., about 0.5 to about 1% (v/v), such as about 1% (v/v)), a ROCK inhibitor, such as Y- 27632 (e.g., about 0.1 to about 0.5% (v/v)), and a combination of EGF and bFGF (e.g., about 0.8% (v/v)). Optionally, the induction step is performed for about 10 to about 12 days.

Optionally, the method further comprises a maturation step which occurs after the expansion step. The maturation step comprises culturing the brain organoid in the presence of vitamin A, a neurotrophic factor, and a growth factor. In various aspects, the neurotrophic factor is brain-derived neurotrophic factor (BDNF) and/or neurotrophin-3 (NT-3). Other neurotrophic factors include, but are not limited to, VEGF, nerve growth factor (NGF), neutrophins, Cerebral dopamine neurotrophic factor (CDNF), Glial cell line-derived neurotrophic factor (GDNF), insulin-like growth factor (IGF), neurotrophin-4 (NT-4), Ciliary neurotrophic factor (CNTF), and Bone morphogenetic protein (BMP). In various aspects, the organoid is cultured in the presence of both BDNF and NT-3. The growth factor is optionally epidermal growth factor (EGF) and/or basic fibroblast growth factor (bFGF), and in some instances the method comprises culturing the organoid in the presence of both EGF and bFGF. As explained above, the medium for the maturation phase may comprise a basic medium matrix with other components suitable for the growth and maturation of neural organoids, such as Neurobasal A™ medium, B-27, PSA, Glutamax, and N-2 supplement. In aspects of the disclosure, the maturation step comprises culturing the brain organoid in the presence of Neurobasal-A™ Medium, about 2% B-27 supplement with vitamin A (v/v), about 1% PSA (v/v) and Glutamax, about 0.5% N-2 supplement (v/v), about 0.2% EGF and/or bFGF (v/v) (e.g., about 0.2% EGF and bFGF), about 0.05% to about 0.1% NT- 3 and/or BDNF (v/v) (corresponding to about 10 to about 50 ng/ml of NT-3 and/or BDNF) (e.g., about 0.05% to about 0.1% NT-3 and BDNF), and/or about 0.05% insulin (v/v), including culturing the organoid in a medium that comprises all of the components together. Optionally, the maturation step is performed for at least about 10 days, such as about 10 to about 20 days, about 10 to about 15 days, or about 10 to about 12 days.

In another aspect, described herein is a method of generating a brain organoid. The method comprises: (i) an induction step comprising culturing adult neural stem cells (NSCs) derived from a subventricular zone (SVZ) in an induction medium comprising a ROCK inhibitor (ROCKi), an N-2 supplement, and a growth factor for a first period of time to produce a neurosphere; (ii) an expansion step comprising culturing the neurosphere in an expansion medium comprising hydrogel matrix (e.g., methylcellulose), extracellular matrix (e.g., Matrigel®), an N-2 supplement, a growth factor, and insulin in a hanging droplet suspension format for a second period of time to produce a brain organoid; and (iii) a maturation step comprising culturing the brain organoid in a maturation medium comprising vitamin A, N-2 supplement, a growth factor, a neurotrophic factor, and insulin for a third period of time.

As described further below, the induction medium, the expansion medium, and the maturation medium may further comprise Neurobasal A™ medium and/or B-27 supplement. Additionally, the growth factor(s) in the induction medium, the expansion medium, and the maturation medium are optionally EGF and bFGF.

With respect to the induction step (also referred to herein as induction phase), the cells are cultured for a first period of time of about 7-14 days, such as about 10-12 days. The induction phase is, in some instances, performed until neurospheres form which are about 100 μm to about 400 μm in size, e.g., about 100 μm to about 300 μm in size or about 200 μm to about 300 μm in size. Optionally, the media is changed every 2-3 days (although this is not required). In various aspects, the induction medium comprises about 0.8% EGF and bFGF (v/v), about 0.5%-1% N-2 supplement (v/v) (e.g., 1%), and about 0.1%-0.5% ROCKi (v/v) (e.g., 0.5%), such as Y-27632. The induction medium may also comprise one or more of (e.g., all of) Neurobasal A™ medium, about 2% B-27 (v/v), and about 1% (v/v) PSA and Glutamax.

With respect to the expansion step, the expansion medium comprises hydrogel matrix (e.g., methylcellulose), extracellular matrix (e.g., Matrigel®), an N-2 supplement, a growth factor, and insulin. The expansion medium does not comprise a ROCKi. The amount of growth factor in the expansion medium is less than the amount of growth factor in the induction media (i.e., there is a reduction in the amount of growth factor applied compared to the induction phase). Also optionally, the expansion medium may comprise an amount of N-2 supplement that is less than that present in the induction medium, although this is not required. Thus, in various embodiments, the amount of growth factor (EGF, bFGF, or a combination of EGF and bFGF) is less than 0.8% in the expansion medium (e.g., between about 0.2% and about 0.6% (v/v), between about 0.3% and 0.5% (v/v), between about 0.2% and 0.4% (v/v), between about 0.4% and about 0.6% (v/v), or about 0.4% (v/v)). In various aspects, the expansion medium comprises about 1% to about 2% extracellular matrix (e.g., Matrigel®) (v/v), about 0.02% hydrogel matrix (e.g., methylcellulose) (w/v), about 0.4% EGF and/or bFGF (v/v) (e.g., about 0.4% EGF and bFGF (v/v)), about 1% N-2 supplement (v/v), and about 0.1% insulin (v/v). The medium also may comprise Neurobasal A™ medium, about 2% B-27 (v/v), and/or about 1% (v/v) PSA and Glutamax, including embodiments where all three components are present in the expansion medium alongside the other components referenced herein.

Also regarding the expansion step, the second period of time is optionally about 10 to about 20 days, e.g., about 10 days. Thus, in reference to the beginning of the method wherein NSCs are introduced into induction media at day 0, neurospheres resulting from the induction phase are introduced into the expansion media and hanging droplet suspension format around day 10-12, and the cells are cultured under the conditions of the expansion phase until about day 18-20. Optionally, between days 14-20, neurosphere fusion is observed. Also optionally, the structures obtained from the expansion phase are about 600 μm to about 900 μm in size before implementing the maturation step. The media is optionally changed every three to four days. In some aspects, extracellular matrix is removed from the culture conditions around 72 hours after initiation of the expansion step (i.e., media exchange is performed with a media which does not comprise extracellular matrix).

Regarding the maturation step, the maturation medium optionally comprises an amount of N-2 supplement that is less than the amount of N-2 supplement present in the expansion media and an amount of growth factor that is less than the amount of growth factor present in the expansion media. For example, the amount of growth factor (e.g., EGF and/or bFGF) may be about 0.1% to about 0.4% (v/v), such as about 0.15% to about 3%, or about 0.2% (v/v). In various aspects, the amount of growth factor is less than 0.4% (v/v). The maturation medium also comprises one or more neurotrophic factors, such as BDNF and/or NT-3 (e.g., about 0.5% to about 0.1% of neurotrophic factor(s), such as BDNF and NT-3). Additionally, the maturation medium may comprise any or all of the components described above. For instance, the maturation medium may comprise about 0.2% EGF (v/v), about 0.2% bFGF (v/v), about 0.05% to about 0.1% BDNF and/or NT-3 (v/v) (e.g., both BDNF and NT-3), about 0.25% to about 0.5% N-2 supplement (v/v), and about 0.05% insulin (v/v). The maturation medium further comprises vitamin A (e.g., B-27 containing vitamin A at about 2% (v/v)). The medium may also comprise Neurobasal A™ medium and about 1% (v/v) PSA and Glutamax. The maturation step also is performed in the hanging drop suspension format.

The disclosure further provides a brain organoid produced by the method described herein, as well as a population of brain organoids produced by the method. Organoids may be characterized using a variety of methods. For instance, brightfield imaging may be employed to consider morphology over time points, assessment of diameter/size, aggregation/sphere-forming capacity, periphery/borders, opacity, and budding. Fluorescence imaging may be utilized to confirm that organoids display marker expression of NSCs, immature, and mature neuronal cells, and some exhibit tissue-like pattern, e. g. neural-tubule-like or presence of single (or multiple) rosettes. The brain organoid optionally exhibits a diameter of about 1 mm (or the population of brain organoids exhibits an average diameter of about 1 mm). In various aspects, the brain organoid exhibits structural properties mimicking that of the neuroepithelium, including the formation of neural rosettes, neuroepithelial budding, and apical to basal polarity (polarized neuroepithelium). Optionally, the brain organoid expresses SOX2, Nestin, GFAP, DCX, MAP2, NeuN, CTIP2, and/or Ki67 (including any combination of the foregoing, such as all of the foregoing).

In various aspects of the disclosure, the brain organoid exhibits properties and/or expresses markers associated with a neural disease or disorder. For instance, the brain organoid may express USP16 or inhibitors of USP16 or downstream makers of the bone morphogenic protein (BMP)-Cdkn2a axis, such as bone morphogenetic protein type 1A (BMPR- 1A) or type 2 receptor (BMPR-2)), which are markers associated with Alzheimer's disease. Alternatively or in addition, the brain organoid may harbor mutant amyloid precursor protein or features of various animal models, such as Tg-SwDI, Tg-APP(ArcSwe), APP/PS1, ARTE10, and/or APP23. In various aspects, the brain organoid mimics Alzheimer's disease-affected brain tissue, although the organoid may mimic features of other neurological or neurodegenerative diseases.

In another aspect, the disclosure provides a method of characterizing the activity of a substance in brain tissue, the method comprising contacting the brain organoid of the disclosure with the substance and characterizing the biological effect of the substance on the brain organoid. The biological effect may comprise biochemical or bioinformatics-related effects. The materials and methods described herein provide improved brain organoids suitable for characterizing disease states and characterizing the biological effect of a substance (e.g., a therapeutic candidate) on brain tissue. Methods of characterizing the activity of a substance on a cell or biological tissue are well known in the art, particularly in the fields of drug discovery and development. For instance, characterizing the activity of a substance with respect to the organoid may include, but is not limited to, detecting or evaluating changes in gene expression in response to the substance, detecting or measuring protein production or changes thereof, including characterizing expression of tissue-or disease-specific markers. Methods of characterizing gene expression and protein expression are well known in the art. Indeed, genomics assays may be employed. The activity of a substance may also be characterized by detecting changes in structural features of the organoid, such a size, morphology, or growth. Alternatively or in addition, metabolomics assays may be performed to examine changes in metabolites triggered by exposure to the substance. The brain organoids of the disclosure allow for high-throughput drug screening, organoid profiling, and rapid single cell resolution phenotyping. Where organoids are generated using patient-derived tissues, the materials and methods described herein provide an improved platform for the development of personalized treatments to neurological disorders. The method is not limited to any particular substance, which may include (but is not limited to), proteins, peptides, small molecules, or other cells.

In yet another aspect, the disclosure provides a kit comprising a container comprising one or more brain organoids of the disclosure in a carrier. A carrier is any solvent, dispersion medium, vehicle, or diluent which is physiologically acceptable for the storage and/or preservation of brain organoids. Examples of carriers include but are not limited to sterile liquids, such as water and oils, including those of petroleum, animal, vegetable, or synthetic origin. Water or aqueous solutions, e.g., saline solutions and aqueous dextrose and glycerol solutions, are also examples of carriers. In various aspects, the brain organoid composition is frozen for storage.

Various aspects of the disclosure are described below:

Aspect 1. A method of generating a brain organoid, the method comprising culturing a neurosphere comprising neural stem cells (NSCs) in a composition comprising from about 0.01 to about 0.1% hydrogel matrix (w/v) and about 1 to about 2% extracellular matrix (ECM) (v/v) in a hanging droplet cell suspension format for a period of time to generate a brain organoid.

Aspect 2. The method of aspect 1, wherein the NSC is derived from a subventricular zone (SVZ).

Aspect 3. The method of aspect 1 or aspect 2, wherein the NSCs are adult NSCs.

Aspect 4. The method of any one of aspects 1-3, wherein the neural stem cells (NSCs) are present within a neurosphere expressing Sox2, Nestin, and GFAP.

Aspect 5. The method of any one of aspects 1-4, wherein the neurosphere is cultured in a hanging droplet suspension in a hanging drop plate coated with hydrophilic material and overlaid on a vessel filled with water.

Aspect 6. The method of any one of aspects 1-5, wherein the hydrogel matrix is methylcellulose and the ECM is Matrigel®, and the composition comprises from about 0.01 to about 0.1% methylcellulose (w/v) and about 1 to about 2% Matrigel (v/v).

Aspect 7. The method of any one of aspects 1-6, wherein the composition further comprises a growth factor and insulin.

Aspect 8. The method of aspect 7, wherein the composition comprises epidermal growth factor (EGF) and basic fibroblast growth factor (bFGF).

Aspect 9. The method of aspect 7 or aspect8, wherein the composition comprises about 0.2 to about 0.8% EGF and bFGF (w/v) and about 0.05 to about 0.1% insulin (v/v).

Aspect 10. The method of any one of aspects 1-9, wherein the period of time is at least about 10 days.

Aspect 11. The method of aspect 10, wherein the period of time is about eight to about 12 days.

Aspect 12. The method of aspect 10, wherein the period of time is about six to about 10 days.

Aspect 13. The method of any one of aspects 1-12, wherein the method further comprises, prior to culturing the neurosphere in the composition, an induction step comprising culturing NSCs in the presence of a ROCK inhibitor (ROCKi) and an N-2 supplement to produce a neurosphere.

Aspect 14. The method of aspect 13, wherein the induction step further comprises culturing the NSCs in the presence of EGF and bFGF.

Aspect 15. The method of aspect 13 or aspect 14, wherein the induction step comprises culturing the NSCs in the presence of Neurobasal A-Medium, about 2% B- 27 supplement (v/v), about 1% PSA (v/v), Glutamax, about 0.5 to about 1% N-2 supplement (v/v), and/or about 0.1 to about 0.5% ROCK inhibitor (v/v).

Aspect 16. The method of any one of aspects 14-16, wherein the induction step is performed for about 10 to about 12 days.

Aspect 17. The method of any one of aspects 1-16, wherein the method further comprises a maturation step comprising culturing the brain organoid in the presence of vitamin A, a neurotrophic factor, and a growth factor.

Aspect 18. The method of aspect 17, wherein the neurotrophic factor is brain-derived neurotrophic factor (BDNF) and/or neurotrophin-3 (NT-3).

Aspect 19. The method of aspect 17 or aspect 18, wherein the growth factor is epidermal growth factor (EGF) and basic fibroblast growth factor (bFGF).

Aspect 20. The method of any one of aspects 17-19,wherein the maturation step comprises culturing the brain organoid in the presence of Neurobasal-A Medium, about 2% B-27 supplement with vitamin A (v/v), about 1% PSA (v/v), Glutamax, about 0.5% N-2 supplement (v/v), about 0.2% EGF and bFGF (w/v), about 0.05 to about 0.1% NT- 3 and/or BDNF (w/v), and/or about 0.05% insulin (v/v).

Aspect 21. The method of any one of aspects 17-20, wherein the maturation step is performed for at least about 10 days.

Aspect 22. The method of aspect 21, wherein the maturation step is performed for about 10 to about 12 days.

Aspect 23. A method of generating a brain organoid, the method comprising: (i) an induction step comprising culturing adult neural stem cells (NSCs) derived from a subventricular zone (SVZ) in an induction medium comprising a ROCK inhibitor (ROCKi), an N-2 supplement, and a growth factor for a first period of time to produce a neurosphere; (ii) an expansion step comprising culturing the neurosphere in an expansion medium comprising hydrogel matrix, extracellular matrix (ECM), an N-2 supplement, a growth factor, and insulin in a hanging droplet suspension format for a second period of time to produce a brain organoid; and (iii) a maturation step comprising culturing the brain organoid in a maturation medium comprising vitamin A, N-2 supplement, a growth factor, a neurotrophic factor, and insulin for a third period of time.

Aspect 24. The method of aspect 23, wherein the induction medium comprises about 0.8% EGF and bFGF (v/v), about 1% N-2 supplement (v/v), and about 0.5% ROCKi (v/v).
Aspect 25. The method of aspect 23 or aspect 24, wherein the first period of time is about 7-14 days.

Aspect 26. The method of any one of aspects 23-25, wherein the induction medium is changed about every two to three days during the first period of time.

Aspect 27. The method of any one of aspects 23-26, wherein the expansion medium comprises an amount of growth factor which is less than the amount of growth factor in the induction media.

Aspect 28. The method of aspect 23, wherein hydrogel matrix of the expansion medium is methylcellulose and the ECM is Matrigel®, and the expansion medium comprises about 1 to about 2% Matrigel (v/v), about 0.01 to about 0.1% methylcellulose (w/v), about 0.4% EGF and bFGF (w/v), about 1% N-2 supplement (v/v), and about 0.1% insulin (v/v).

Aspect 29. The method of any one of aspects 23-28, wherein the second period of time is about 10 to about 20 days.

Aspect 30. The method of aspect 29, wherein the second period of time in the expansion step (ii) is about 10 days.

Aspect 31. The method of any one of aspects 23-30, wherein the expansion medium is changed about every three to four days during the second period of time.

Aspect 32. The method of any one of aspects 23-31, wherein the maturation medium comprises an amount of N-2 supplement that is less than the amount of N-2 supplement present in the expansion media and an amount of growth factor that is less than the amount of growth factor present in the expansion media.

Aspect 33. The method of any one of aspects 23-32, wherein the maturation medium comprises (i) about 0.2% EGF (w/v), (ii) about 0.2% bFGF (v/v), (iii) about 0.05 to about 0.1% BDNF and/or NT-3 (w/v), (iv) about 0.25 to about 0.5% N-2 supplement (v/v), and (v) about 0.05% insulin (v/v).

Aspect 34. The method of any one of aspects 23-33, wherein the induction medium, the expansion medium, and the maturation medium further comprise Neurobasal-A Medium.

Aspect 35. The method of any one of aspects 23-34, wherein the growth factor(s) in the induction medium, the expansion medium, and the maturation medium are EGF and bFGF.

Aspect 36. The method of any one of aspects 23-35, wherein the induction medium, the expansion medium, and the maturation medium further comprise B-27 supplement.

Aspect 37. The method of any one of aspects 1-36, wherein the NSCs are derived from a subject suffering from a neurological disease or disorder.

Aspect 38. The method of aspect 37, wherein the neurological disease or disorder is an Alzheimer's disease.

Aspect 39. A brain organoid produced by the method of any one of aspects 1-38.

Aspect 40. The brain organoid of aspect 39, having a diameter of about 1 mm.

Aspect 41. The brain organoid of aspect 39 or aspect 40, expressing SOX2, Nestin, GFAP, DCX, MAP2, NeuN, CTIP2, and/or Ki67.

Aspect 42. A method of characterizing the activity of a substance in brain tissue, the method comprising contacting the brain organoid of any one of aspects 39-41 with the substance and characterizing the biological effect of the substance on the brain organoid.

Aspect 43. The method of aspect 42, wherein the brain organoid mimics Alzheimer's disease-affected brain tissue.

Aspect 44. A kit comprising a container comprising one or more brain organoids of any one of aspects 39-42 in a carrier.

### EXAMPLES

The examples are offered for illustrative purposes only and are not intended to limit the scope of the present invention in any way.

### Example 1

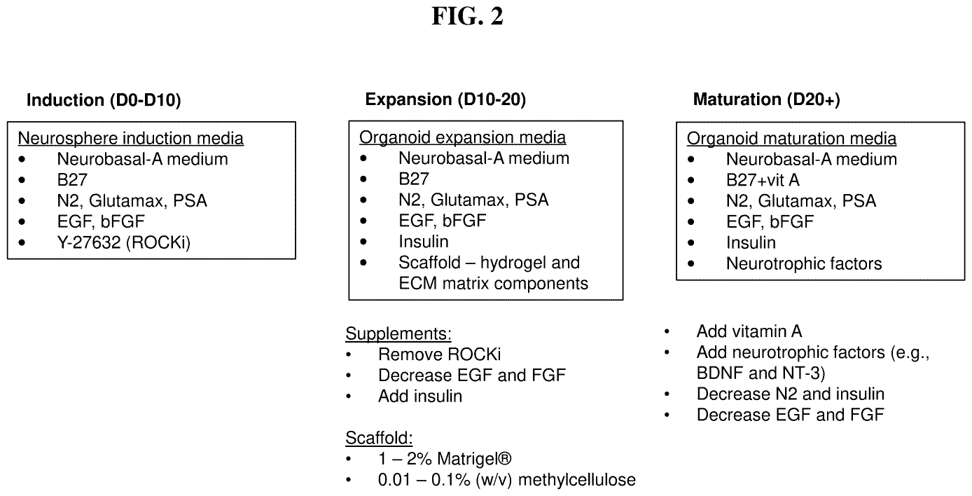

This example describes a representative method of producing brain organoids of the disclosure. For ease of reference, the method is described in terms of (i) neurosphere induction, (ii) organoid expansion, and (iii) maturation. Various aspects of the method are illustrated in FIG. 1. The media used in the various stages of organoid production described below are illustrated in FIG. 2.

Induction: Briefly, an excision of the SVZ with micro-dissection techniques was performed on 3-month-old wild-type mice. The NSCs were cultured in 6-well plates with the neurosphere induction media described herein (including N-2 supplement and ROCKi), and allowed to expand from days 0-10 (i.e., D0-D10) until reaching about 200-500 μm.

Expansion: To overcome previous hurdles of organoid technology, a 3D spheroid protocol was developed. The organoid platform utilized a high-throughput 384-well hanging droplet plate sandwiched between a standard 96-well plate filled with distilled water to provide a humidified environment and prevent evaporation of small volume droplets. Using a hydrophilic coating of the 384-well plate, droplets were easily dispensed (25 μl) through the access holes, which holds a cell suspension droplet for long term use. The platform is amenable to use with a liquid handling robot for automated cell culture techniques and media changes.

After day 10 (D10), intact neurospheres were transferred directly into a 384-well hanging droplet system using wide-bore pipette tips and were deposited in small volume of 25 μl droplets. A custom expansion medium was prepared to mimic the biophysical properties of the brain tissue microenvironment. Methylcellulose (˜0.02% (w/v) MethoCel™) and Matrigel® (1-2%) provided an ideal scaffold microenvironment for brain organoid formation. The expansion medium also comprised growth factors, EGF and bFGF, but in a reduced amount compared to the induction medium. The expansion medium also included insulin, as well as N-2 supplement. After 72 hours, Matrigel® was removed. Media was replenished every 2-3 days, and the development of 3D structures was monitored.

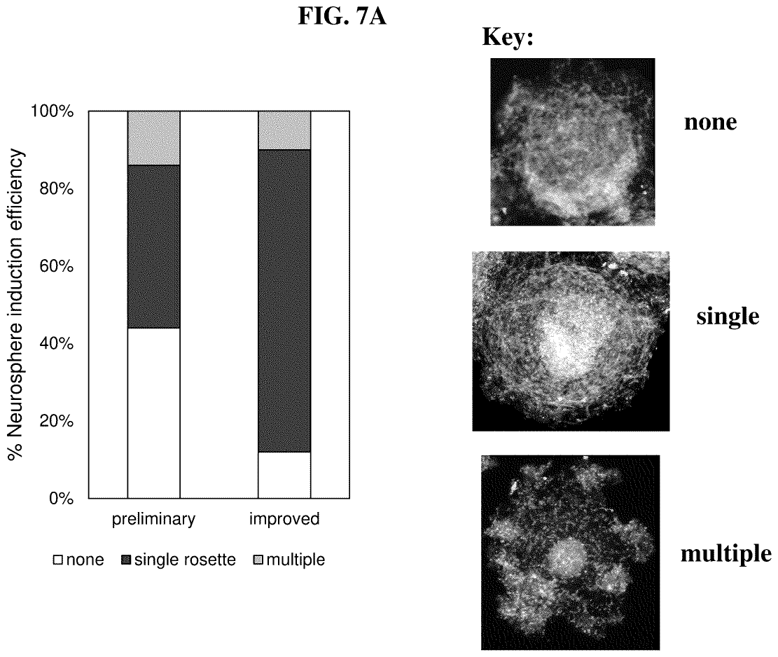

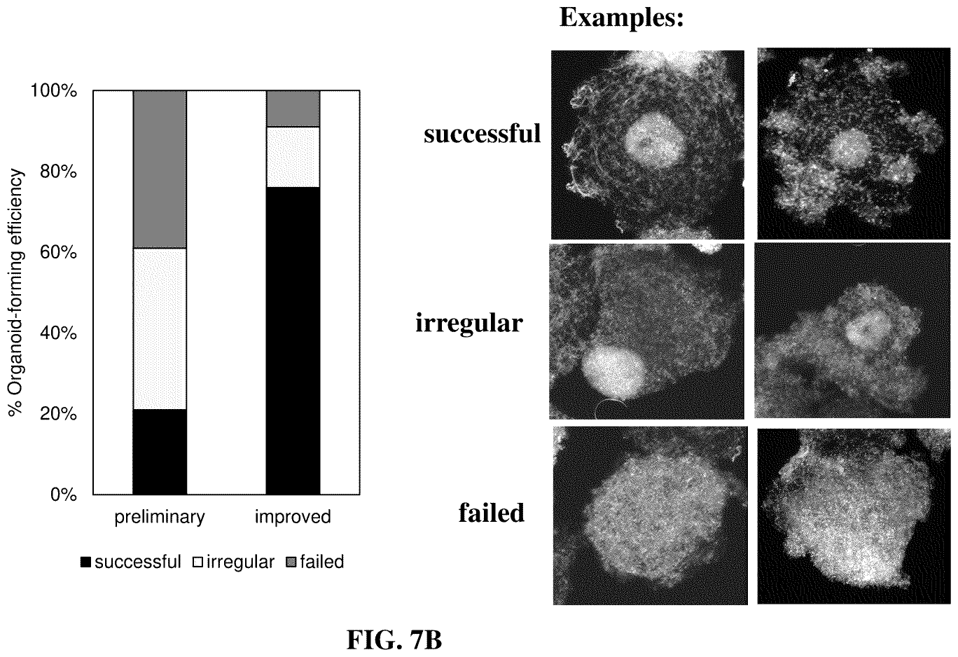

It was determined that neurospheres at least 100 μm in size provided the best results as far as successful expansion from neurosphere to organoid during the expansion phase. Neurospheres which were smaller in size did not expand sufficiently to reproducibly achieve organoids of the desired quality. While neurospheres at least 100 μm in size achieved a sufficient level of expansion and development during the expansion phase, neurospheres between about 200 μm and 300 μm achieved the best results. See also Example 3 and FIGS. 7A and 7B.

Maturation: Once organoids displayed features of the neuroepithelium, such as neuroepithelial budding (e.g., opacity, radial growths or projections) and achieved a diameter of about 600-900 μm (about day 18-20), the expansion media was exchanged with media formulated to promote brain organoid maturation. The maturation medium comprised further reduced amounts of growth factor, as well as reduced amounts of N-2 supplement and insulin compared to the expansion medium. The maturation medium further comprises vitamin A and neurotrophic factors, here BDNF and NT-3. The organoids were cultured in the maturation medium for 10-12 days. Features of the resulting organoids are described below.

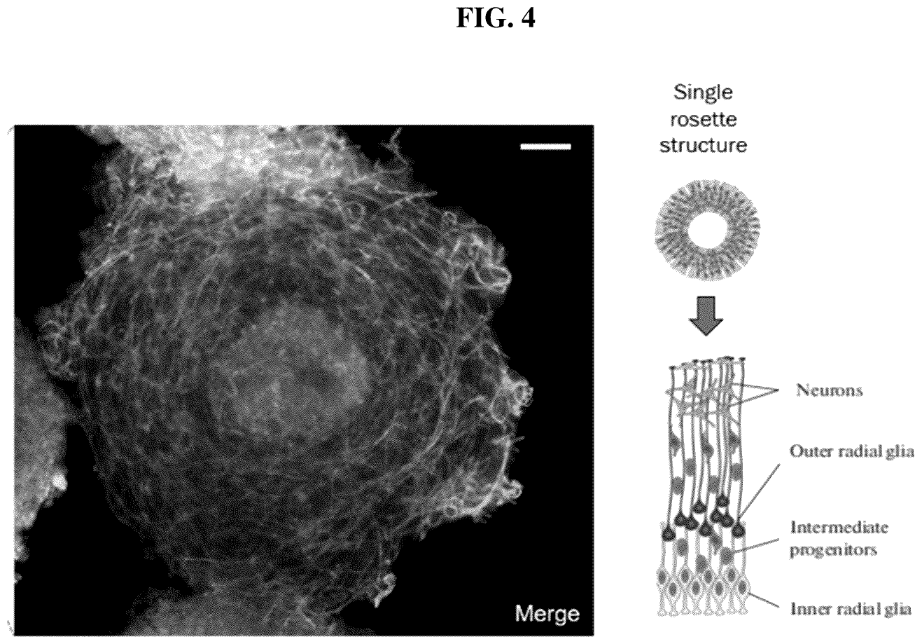

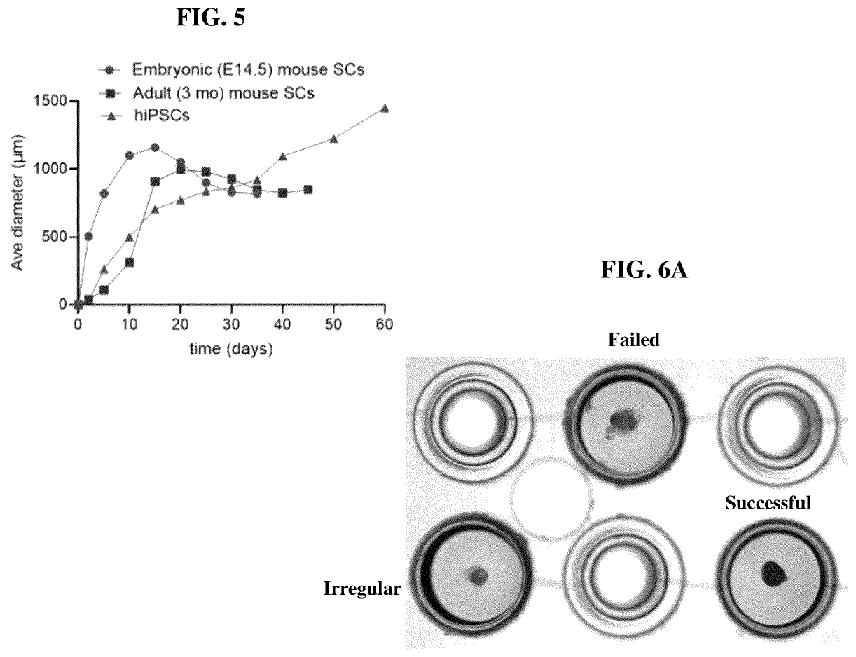

This example, for the first time, demonstrates that basic features of a 3D neuroepithelium derived from adult NPCs is possible, and that these structures can expand to form defined progenitor zones (FIG. 4). Moreover, the study showed evidence of neuroepithelial buds based on their morphology (FIG. 1, images) and expression patterns exhibiting a region specific, layered organization from immunofluorescence imaging of canonical neuronal markers. These included neural stem or progenitor cells (SOX2, Nestin, and GFAP), immature neurons (DCX), mature neurons (MAP2, NeuN), deep cortical layer neurons (CTIP2), and proliferation marker (Ki67). The study also demonstrated that NSCs derived from murine SVZ can develop both single and multiple neural rosette-like structures, showing both immature and mature layers consistent with the in vivo cortical plate, inside-out patterning of the human cortex. Morphology and size of the organoids was tracked over 50 days in 3D culture and across passage number. A maximal average diameter of ˜1mm was observed at day 20. These results were compared previous studies using other cell types. The adult NSCs derived from SV and cultured in the instant method (FIG. 5, squares) required reduced culture time to achieve a desired size compared to iPSC-derived brain organoids cultured in previous methodologies (FIG. 5, triangles). The ability to reproducibly generate organoids with a single rosette structure is a significant advantage of the instant method over previously disclosed methods.

### Example 2

In various aspects, the disclosure provides a method comprising (i) an induction step comprising culturing adult neural stem cells (NSCs) derived from a subventricular zone (SVZ) in an induction medium comprising a ROCK inhibitor (ROCKi), an N-2 supplement, and a growth factor for a first period of time to produce a neurosphere; (ii) an expansion step comprising culturing the neurosphere in an expansion medium comprising hydrogel matrix, extracellular matrix (ECM), an N-2 supplement, a growth factor, and insulin in a hanging droplet suspension format for a second period of time to produce a brain organoid; and (iii) a maturation step comprising culturing the brain organoid in a maturation medium comprising vitamin A, N-2 supplement, a growth factor, a neurotrophic factor, and insulin for a third period of time. This example describes the testing of various parameters of the brain organoid method described herein.

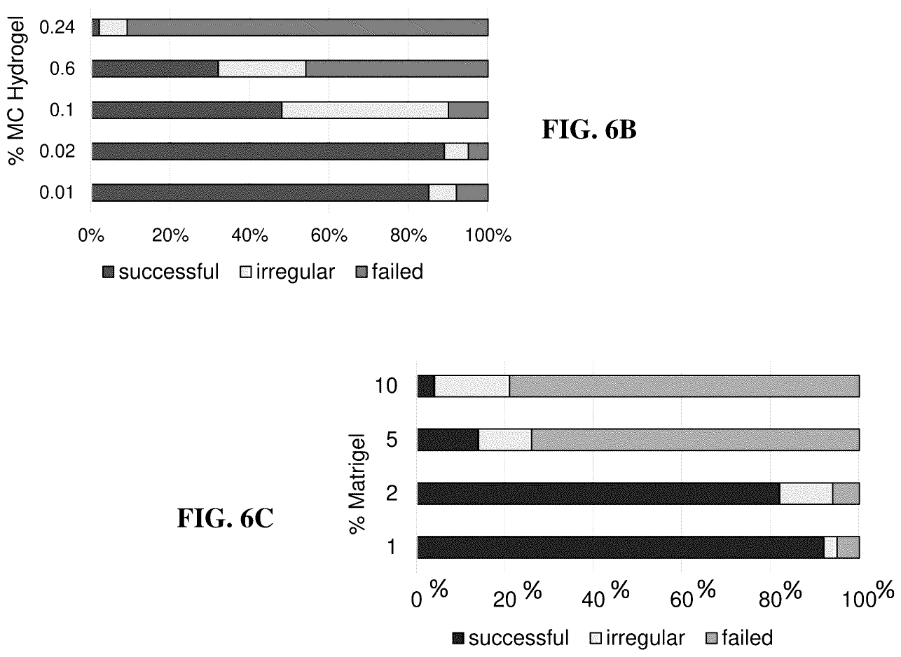

The amounts of hydrogel (here, methylcellulose) and extracellular matrix (here, Matrigel®) were varied and the effect on organoid formation was characterized using a hanging drop array plate. FIGS. 6A-6C illustrate representative results from the study. Between about 0.01% and 0.1% methylcellulose resulted in at least about 40% successful organoids produced, with 0.01% and 0.02% offering better results of an over 80% success rate. Between about 1% and 2% Matrigel® resulted in at least about 80% successful organoids produced. Higher amounts of hydrogel and extracellular matrix were linked with increased failure rates. Irregular or failed results were marked by reduced cell viability and survival and improper organoid formation, seen by excessive outgrowths and a “halo effect” around the organoid when viewed microscopically. Successful organoid formation was marked by, e.g., well-defined, smooth, round organoid boundaries, neuroepithelial budding, opaque features, optically translucence with clear radial organization, and lack of cysts or cell processes.

### Example 3

This example describes studies characterizing the effect of different components on the method disclosed herein.

Briefly, excision of the SVZ was performed with micro-dissection and/or surgical techniques from three-month old wild-type mice, and the neural stem cell (NSC) niche was harvested. The NSCs were grown in non-treated tissue culture six-well plates with neurosphere induction media (including N2 and ROCKi supplements), and were allowed to expand from days 0-10 until they reached a size of˜200-500 μm. It was observed that culture in the growth media described herein (neurosphere induction) prior to expansion into organoids achieved improved results, with an average diameter of modified versus pre-optimized neurospheres to be 250±75 μm and 110±40 μm, respectively.

For the expansion phase after day 10, intact neurospheres were transferred directly into a 384-well hanging droplet system using wide-bore pipette tips and depositing them in small volume 25 μl droplets. For the organoid expansion media, a custom extracellular matrix was prepared that mimics the biophysical properties of the brain tissue microenvironment, using a hybrid biomaterial composite formulation designed for the expansion of neural stem cells. This included an ultra-low concentration of the macromolecular additive methylcellulose at ˜0.02% (w/v) (MethoCel (MC)) together with a low concentration of dissolved Matrigel (1-2%, MG).

For the expansion media additives, EGF and bFGF levels were decreased and insulin supplement was added. After 72 hrs, Matrigel was removed, while fresh media was replenished every 2-3 days. 3D structures in vitro were monitored. It was observed that expanded organoids begin to display neural-like features both morphologically and phenotypically, and that a threshold between 0.01-0.02 (w/v) % methylcellulose and 1-2 (v/v)% Matrigel was sufficient, with >80% forming successful organoids. However, higher concentrations of both MC and MG led to >50% irregular or failed organoids. Interestingly, the expansion period yielded an exponential type growth rate, where average organoid diameter increased 2.8×+1.2 with a range of 1.5-5×, while ESC or iPSC-derived organoids typically display a logarithmic growth during neural induction and expansion.

For the next phase, the media was switched to organoid maturation media. Notably, this phase included further decreasing the supplements N2, EGF, and bFGF, and the addition of vitamin A and neurotrophic factors BDNF, and NT-3. The small droplet diffusion kinetics produces an ideal symmetry for both the expansion and maturation phases due to improved oxygen and nutrient exchange without the need for additional equipment for agitation of 3D structures, such as using a spinning bioreactor. At maturation, organoids displayed clearly observable features of the neuroepithelium, such as neuroepithelial budding (e.g., with radial growths or projections), opacity, and thicker walls, and achieved desired diameter (˜600-1200 μm) after day 18-20. By fluorescence, brain organoids with a more differentiated state exhibit single or multiple rosette structures and are positive for the expression of earlier to later differentiation markers, namely neural stem cell (SOX2, GFAP, and Nestin), immature (DCX), mature (NeuN, Map2) and deep cortical (Ctip2) markers. The presence of budding is indicative of the stem cell niche that controls neural stem cell renewal and homeostasis, and can be used to study disease progression, differentiation and optimize the ECM and neuroepithelial patterning.

An advantage of the high-throughput single organoid-per-droplet setting described herein is that the uniform radial organoid growth and budding pattern are highly reproducible and deliver faster quantitative results. Subsequently, organoids can be easily collected for downstream quantitative testing, such as multi-omics analyses or biochemical assays without requiring complicated procedures. Across all phases of organoid formation, the study introduced quantifiable parameters called “% neurosphere-to-expansion efficiency” and the “overall % organoid-forming efficiency.” It was determined from preliminary results using other methodology that a large portion of roughly 80% produced either irregular or failed organoids, and despite 55% of the initial organoids being able to form a neural-like pattern, they displayed the presence of undesirable effects such as cell debris/death, irregular patterning, incorrect radial growth, low viability, and lack of budding. In contrast, the protocol described herein resulted in >80% successfully formed organoids, where only 10-12% failed to exhibit any differentiation or neural rosette pattern.

To summarize, the following qualitative and quantitative features were examined: morphology from brightfield imaging based on neurosphere-forming capacity, diameter, thin/thick walls, opacity from center towards periphery, budding, fluorescence results, positive expression of the aforementioned early to late differentiation markers, and broad neuroepithelial patterning. The improved methodology described herein resulted in improved organoid forming efficiency and improved organoid expansion efficiency.

All references, including publications, patent applications, and patents, cited herein are hereby incorporated by reference to the same extent as if each reference were individually and specifically indicated to be incorporated by reference and were set forth in its entirety herein.

1. A method of generating a brain organoid, the method comprising culturing a neurosphere comprising neural stem cells (NSCs) in a composition comprising from about 0.01 to about 0.1% hydrogel matrix (w/v) and about 1 to about 2% extracellular matrix (ECM) (v/v) in a hanging droplet cell suspension format for a period of time to generate a brain organoid.

2. The method of claim 1, wherein the NSC is derived from a subventricular zone (SVZ).

3. (canceled)

4. The method of claim 1, wherein the neural stem cells (NSCs) are present within a neurosphere expressing Sox2, Nestin, and GFAP.

5. (canceled)

6. The method of claim 1, wherein the hydrogel matrix is methylcellulose and the ECM is Matrigel®, and the composition comprises from about 0.01 to about 0.1% methylcellulose (w/v) and about 1 to about 2% Matrigel (v/v).

7. The method of claim 1, wherein the composition further comprises epidermal growth factor (EGF) and/or basic fibroblast growth factor (bFGF), and insulin.

8. (canceled)

9. The method of claim 7, wherein the composition comprises about 0.2 to about 0.8% EGF and bFGF (w/v) and about 0.05 to about 0.1% insulin (v/v).

10. The method of claim 1, wherein the period of time is at least about 10 days.

11. The method of claim 1, wherein the period of time is about eight to about 12 days.

12. The method of claim 1, wherein the period of time is about six to about 10 days.

13. The method of claim 1, wherein the method further comprises, prior to culturing the neurosphere in the composition, an induction step comprising culturing NSCs in the presence of a ROCK inhibitor (ROCKi) and an N-2 supplement to produce a neurosphere.

14. The method of claim 13, wherein the induction step further comprises culturing the NSCs in the presence of EGF and bFGF.

15. The method of claim 13, wherein the induction step comprises culturing the NSCs in the presence of Neurobasal A-Medium, about 2% B- 27 supplement (v/v), about 1% PSA (v/v), Glutamax, about 0.5 to about 1% N-2 supplement (v/v), and/or about 0.1 to about 0.5% ROCK inhibitor (v/v).

16. The method of claim 14, wherein the induction step is performed for about 10 to about 12 days.

17. The method of claim 1, wherein the method further comprises a maturation step comprising culturing the brain organoid in the presence of (i) vitamin A, (ii) brain-derived neurotrophic factor (BDNF) and/or neurotrophin-3 (NT-3), and (iii) EGF and/or bFGF.

18-19. (canceled)

20. The method of claim 17, wherein the maturation step comprises culturing the brain organoid in the presence of Neurobasal-A Medium, about 2% B-27 supplement with vitamin A (v/v), about 1% PSA (v/v), Glutamax, about 0.5% N-2 supplement (v/v), about 0.2% EGF and bFGF (w/v), about 0.05 to about 0.1% NT-3 and/or BDNF (w/v), and/or about 0.05% insulin (v/v).

21. The method of claim 17, wherein the maturation step is performed for at least about 10 days.

22. (canceled)

23. A method of generating a brain organoid, the method comprising:
(i) an induction step comprising culturing adult neural stem cells (NSCs) derived from a subventricular zone (SVZ) in an induction medium comprising a ROCK inhibitor (ROCKi), an N-2 supplement, and EGF and/or bFGE for a first period of time to produce a neurosphere;
(ii) an expansion step comprising culturing the neurosphere in an expansion medium comprising hydrogel matrix, extracellular matrix (ECM), an N-2 supplement, EGF and/or bFGF in an amount that is less than the amount of EGF and/or bFGF in the induction medium, and insulin in a hanging droplet suspension format for a second period of time to produce a brain organoid; and
(iii) a maturation step comprising culturing the brain organoid in a maturation medium comprising vitamin A, N-2 supplement in an amount that is lower than the amount of N-2 supplement in the expansion medium, EGF and/or bFGF, BDNF and/or NT-3, and insulin for a third period of time.

24. (canceled)

25. The method of claim 23, wherein the first period of time is about 7-14 days, and the second period is about 10 to 20 days.

26. The method of claim 23, wherein the induction medium is changed about every two to three days during the first period of time, and the expansion medium is changed every three to four days during the second period.

27. (canceled)

28. The method of claim 23, wherein hydrogel matrix of the expansion medium is methylcellulose and the ECM is Matrigel®, and the expansion medium comprises about 1 to about 2% Matrigel (v/v), about 0.01 to about 0.1% methylcellulose (w/v), about 0.4% EGF and bFGF (w/v), about 1% N-2 supplement (v/v), and about 0.1% insulin (v/v).

29-32. (canceled)

33. The method of claim 23, wherein the maturation medium comprises (i) about 0.2% EGF (w/v), (ii) about 0.2% bFGF (v/v), (iii) about 0.05 to about 0.1% BDNF and/or NT-3 (w/v), (iv) about 0.25 to about 0.5% N-2 supplement (v/v), and (v) about 0.05% insulin (v/v).

34. The method of claim 23, wherein the induction medium, the expansion medium, and the maturation medium further comprise Neurobasal-A Medium and B-27 supplement.

35-36. (canceled)

37. The method of claim 1, wherein the NSCs are derived from a subject suffering from a neurological disease or disorder.

38. (canceled)

39. A brain organoid produced by the method of claim 1, wherein the brain organoid has a diameter of about 1 mm and/or expresses one or more markers selected from SOX2, Nestin, GFAP, DCX, MAP2, NeuN, CTIP2, and/or Ki67.

40-41. (canceled)

42. A method of characterizing the activity of a substance in brain tissue, the method comprising contacting the brain organoid of claim 39 with the substance and characterizing the biological effect of the substance on the brain organoid.

43-44. (canceled)
